(19) United States
(12) Patent Application Publication
(10) Pub. No.: US 2025/0000835 A1
(43) Pub. Date: Jan. 2, 2025

(54) SYNERGIES OF 1- BETA-CARYOPHYLLENE, CANNABIDIOL AND TRIBUTYRIN; 2- RETINYL PALMITATE, BETA-CARYOPHYLLENE AND CANNABIDIOL; 3- RETINYL PALMITATE AND BETA-CARYOPHYLLENE IN CONTROLING INFLAMMATION, METABOLIC HEALTH, INSULIN SENSITIVITY/ GLYCEMIC - GLUCOSE UPTAKE & MANAGEMENT, AND FATTY LIVER/ NAFLD

(71) Applicant: FLG Sciences, LLC, Miramar, FL (US)

(72) Inventors: Marcello Ferro, Miramar, FL (US); Adolfo Graubard, Weston, FL (US); Hector L. Lopez, Cream Ridge, NJ (US); Timothy N. Ziegenfuss, Chardon, OH (US)

(21) Appl. No.: 18/763,849

(22) Filed: Jul. 3, 2024

Related U.S. Application Data
(63) Continuation of application No. PCT/US2022/054229, filed on Dec. 29, 2022.
(60) Provisional application No. 63/296,642, filed on Jan. 5, 2022.

(51) Int. Cl. A61K 31/22; A61K 31/015; A61K 31/07; A61P 39/06

(52) U.S. Cl. CPC A61K 31/22; A61K 31/015; A61K 31/07; A61P 39/06

(57) ABSTRACT

Disclosed here is the method of treating general inflammation, metabolic health/insulin resistance/glycemic-glucose uptake & management (affecting skeletal muscle, liver, adipose, skin, gut, fibro-connective/joint, kidney, brain/CNS and other tissues), systemic inflammation, Immuno-modulation & Metabolic Syndrome/Nonalcoholic fatty liver disease (NAFLD), and provide Immuno-modulation, Insulin Sensitizing and Immuno-Metabolic benefits with at least one short fatty acid, such as butyrate or butyric acid in form of tributyrin, used in conjunction with one cannabinoid, such as cannabidiol or CBD, and with at least one terpene, such as beta-caryophyllene or BCP.

### BACKGROUND

The present invention relates the pharmaceutical potential of 3 different formulas: 1-beta-caryophyllene plus cannabidiol and tributyrin; 2-Retinyl palmitate (Vit A) plus beta-caryophyllene and cannabidiol; 3-Retinyl palmitate (Vit A) plus beta-caryophyllene in controlling inflammation markers, apoptosis as well as enhance cellular antioxidant activity. More specifically, the pharmaceutical compositions treat metabolic health/insulin resistance/glycemic-glucose uptake & management/metabolic syndrome (affecting multiple tissues and organ systems such as: skeletal muscle, liver, adipose, dermis/epidermis, connective tissues, renal, gastrointestinal/gut and brain/CNS), InflammationNonalcoholic fatty liver disease (NAFLD), and provide Immuno-modulatory and Immuno-Metabolic benefits. The metabolic syndrome is characterized by a group of metabolic risk factors in the same person (Aging Dis. 2015 March; 6 (2): 109-120. Bonomini F. et al). According to the same authors, the first unified agreement about the definition of the Metabolic Syndrome (MS) was drawn up during a meeting organized by the International Diabetes Federation (IDF) in 2005. One of the defects in MS and its associated diseases is excess cellular oxidative stress (reactive oxygen and nitrogen species, ROS/RNS), and oxidative damage to mitochondrial components, resulting in reduced efficiency of the electron transport chain (J Cell Biochem. 2007 Apr. 15; 100 (6): 1352-69. Nicolson G. L.) The present invention attempts to solve these problems as well as others.

### SUMMARY OF THE INVENTION

Provided herein are methods and compositions comprising: 1-beta-caryophyllene plus cannabidiol and tributyrin; 2-Retinyl palmitate (Vit A) plus beta-caryophyllene and cannabidiol; 3-Retinyl palmitate (Vit A) plus beta-caryophyllene in controlling inflammation markers, apoptosis as well as enhance cellular antioxidant activity. More specifically, the pharmaceutical compositions treat metabolic health/insulin resistance/glycemic-glucose uptake & management/metabolic syndrome (affecting multiple tissues and organ systems such as: skeletal muscle, liver, adipose, dermis/epidermis, connective tissues, renal, gastrointestinal/gut and brain/CNS), InflammationNonalcoholic fatty liver disease (NAFLD), and provide Immuno-modulatory and Immuno-Metabolic benefits.

The methods and compositions are set forth in part in the description which follows, and in part will be obvious from the description, or can be learned by practice of the methods and compositions The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1A is a graph showing the Cellular Antioxidant (AOx) Capacity in AML-12 liver cells of cannabidiol (CBD), tributyrin (Tribut); 2-Retinyl palmitate (Vit A), beta-caryophyllene (β-Caryp); FIG. 1B is a graph showing the Cellular AOx Capacity in AML-12 liver cells of Vit A-beta-caryophyllene (βC)-tributyrin (TriB), Vit A-βC-CBD, βC-CBD-TriB, and Vit A-βC; FIG. 1C is a graph showing the Cellular AOx Capacity in C2C12 muscle cells of CBD, Vit A, Tribut, and β-Caryp; FIG. 1D is a graph showing the Cellular AOx Capacity in C2C12 muscle of Vit A-βC-TriB, Vit A-βC-CBD, βC-CBD-TriB, and Vit A-βC; where all treatments were compared back to the CTL cells using dependent sample t-tests; no comparison between 3-hr versus 12-hr treatments were made; * indicates greater than control cells; and # indicates less than control cells.

FIG. 2A is a graph showing SOD1 protein levels of the AML-12 liver cells of Vit A-βC-TriB, Vit A-βC-CBD, βC-CBD-TriB, and Vit A-βC; FIG. 2B is a graph showing SOD1 protein levels of the C2CL12 liver cells of Vit A-βC-TriB, Vit A-βC-CBD, βC-CBD-TriB, and Vit A-βC; all treatments were compared back to the CTL cells using dependent samples t-tests; no comparison between 3-hr versus 12-hr treatments were made; *, indicates greater than control cells; #, indicates less than control cells.

FIG. 3A is a graph showing SOD2 protein levels in the AML-12 liver cells tested with CBD, Vit A, Tribut, and β-Caryp: FIG. 3B is a graph showing SOD2 protein levels in the C2CD12 muscle cells tested with Vit A-βC-TriB, Vit A-βC-CBD, βC-CBD-TriB, and Vit A-βC; FIG. 3C is a graph showing SOD2 protein levels in the C2CD12 muscle cells tested with CBD, Vit A, Tribut, and β-Caryp; FIG. 3D is a graph showing SOD2 protein levels in the C2CD12 muscle cells tested with Vit A-βC-TriB, Vit A-βC-CBD, βC-CBD-TriB, and Vit A-βC; all treatments were compared back to the CTL cells using dependent samples t-tests; no comparison between 3-hr versus 12-hr treatments were made; and *, indicates greater than control cells; #, indicates less than control cells.

FIG. 4A is a graph showing PPARγ DNA binding activity in AML-12 liver cells tested with CBD, Vit A, Tribut, and β-Caryp; FIG. 4B is a graph showing PPARγ DNA binding activity in AML-12 liver cells tested with Vit A-βC-TriB, Vit A-βC-CBD, βC-CBD-TriB, and Vit A-βC; FIG. 4C is a graph showing PPARγ DNA binding activity in C2C12 muscle cells tested with CBD, Vit A, Tribut, and β-Caryp; FIG. 4D is a graph showing PPARγ DNA binding activity in AML-12 liver cells tested with Vit A-βC-TriB, Vit A-βC-CBD, βC-CBD-TriB, and Vit A-βC; all treatments were compared back to the CTL cells using dependent samples t-tests; no comparison between 3-hr versus 12-hr treatments were made; *, indicates greater than control cells; #, indicates less than control cells.

FIG. 5A is a graph showing the Inflammatory signaling (pro-caspase 1) in AML-12 liver cells tested with Vit A-βC-TriB, Vit A-βC-CBD, βC-CBD-TriB, and Vit A-βC; FIG. 5B is a graph showing the Inflammatory signaling (pro-caspase 1) in C2CL12 muscle cells tested with Vit A-βC-TriB, Vit A-βC-CBD, βC-CBD-TriB, and Vit A-βC; notably, all treatments were compared back to the CTL cells using dependent samples t-tests; no comparison between 3-hr versus 12-hr treatments were made; #, indicates less than control cells.

FIG. 6A is a graph showing the Inflammatory signaling (phosphorylated NF-kB) in AML-12 liver cells tested with CBD, Vit A, Tribut, and β-Caryp; FIG. 6B is a graph showing the Inflammatory signaling (phosphorylated NF-kB) in AML-12 liver cells tested with Vit A-βC-TriB, Vit A-βC-CBD, βC-CBD-TriB, and Vit A-βC; FIG. 6C is a graph showing the Inflammatory signaling (phosphorylated NF-kB) in C2CL12 muscle cells tested with CBD, Vit A, Tribut, and β-Caryp; FIG. 6D is a graph showing the Inflammatory signaling (phosphorylated NF-kB) in C2CL12 muscle cells tested with Vit A-βC-TriB, Vit A-βC-CBD, βC-CBD-TriB, and Vit A-βC; notably, all treatments were compared back to the CTL cells using dependent samples t-tests; no comparison between 3-hr versus 12-hr treatments were made; *, indicates greater than control cells; #, indicates less than control cells.

FIG. 7A is a graph showing Cellular glucose levels in AML-12 liver cells tested with CBD, Vit A, Tribut, and β-Caryp; FIG. 7B is a graph showing Cellular glucose levels in AML-12 liver cells tested with Vit A-βC-TriB, Vit A-βC-CBD, βC-CBD-TriB, and Vit A-βC; FIG. 7C is a graph showing Cellular glucose levels in C2C12 muscle cells tested with CBD, Vit A, Tribut, and β-Caryp; FIG. 7D is a graph showing Cellular glucose levels in C2C12 muscle cells tested with Vit A-βC-TriB, Vit A-βC-CBD, βC-CBD-TriB, and Vit A-βC; all treatments were compared back to the CTL cells using dependent samples t-tests; no comparison between 3-hr versus 12-hr treatments were made; *, indicates greater than control cells; #, indicates less than control cells.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

The term “synergistic” as used herein is refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-inflammatory; ingredient B is pro-inflammatory; when A and B are combined, said combination produces an anti-inflammatory effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. Pharmaceutical composition includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes).). Exemplary prodrugs release the corresponding free acid upon cleavage, and the hydrolyzable ester-forming residues of the compounds of the present invention.

The term “pharmaceutically acceptable” refers to derivatives, analogues and salts which are physiologically acceptable for use in mammals, and which are not unduly toxic or otherwise unacceptable for such use. The term “mammals” includes human and non-human mammals, including domestic animals, e.g. cats, dogs, rodents, cattle, horses and the like, as well as non-domesticated animals.

The term “excipient” as used herein has its conventional meaning and refers to a pharmaceutically acceptable ingredient, which is commonly used in the pharmaceutical technology for preparing a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes. The term “veterinary composition” encompasses the full range of compositions for internal administration and feeds and drinks which can be consumed by animals.

### Beta-caryophyllene

The term “beta-caryophyllene” is used herein to encompass the secondary metabolite, bicyclic sesquiterpene,-4, 1 1,11-trimethyl-8-methylene-bicyclo[7.2.0]undec-4-ene, which is present in, for example, plant-derived oleoresins, essential oils, solutes, distillates, extracts, fermentations, infusions and leaching, from plants including, but not limited to, Cannabis spp. including Cannabis sativa, Cannabis indica and Cannabis ruderalis, Humulus lupulus, Carum nigrum, Eugenia caryophyllata, Ocimum micranthum, Origanum vulgare, Piper guineense, Cinnamomum zeylanicum, Carthamus tinctorius, Helichrysum italicum, Copaifera spp. including Copaiba officinalis, Copaibaguianensis, Copaiba martii hayne, Copaiba Duckei, Copaiba reticulata, Copaiba multijuga, Copaiba confertiflora, Copaiba langsdorffii, Copaiba coriacea, Copaiba trapezifolia, Copaiba lucens, Copaiba paupera and Copaiba cearensis, Syzygium aromaticum (clove), piper nigrum (black pepper), Zingiber nimmoni, Zingiber officinale, Ocimum canum, Ocimum selloi, Piper cubeba, Aframomum melegueta, Panax ginseng, Zanthoxylum piperitum, Zanthoxylum simulans, Zanthoxylum bungeanum, Zanthoxylum rhesta, Zanthoxylum acanthopodium, Zanthoxylum piperitum, Syzgium aromaticum, Mentha longifolia, Ocimum tenuiflorum, Micromeria fruticosa, Salvia triloba, Salvia canariensis, Rosmarinus officinalis, Satureja thymbra, Satureja montana, Micromeria fruticosa subsp. Barbata, Piper longum, Piper retrofractum, Satureja obovata, Schinus terebinthifolius, Spilanthes acmella, Spilanthes oleracea, Persicaria hydropiper, Artemisia abrotanum, Persicaria odorata, Rhus coriaria, Xylopia aethiopica, Cymbopogon citratis, Pandanus amaryllifolius, Myrica gale, Myrica cerifera, Myrica pensylvanica, Origanum heracleoticum, Ocimum kilimandscharicum, Melissa officinalis, Mentha acquatica, Salvia officinalis, Salvia gillesii, Hyssopus officinalis, Thymus vulgaris, Teucrium cyprium, Teucrium divaricatum var. canescens, Artemisia salsoloides, Thymus zygis subsp. Sylvestris, Teucrium chamaedrys, Origanum minutiflorum, Ocimum basilicum, Thymus x citriodorus, Micromeria Juliana, Origanum onites, Origanum vulgare subsp. hirtum, Rosmarinus tomentosus, Lippia alba, Thymus riatarum, Rosemarinus eriocalyx, Ageratum conyzoides, Teucrium arduini, Teucrium kotschyanum, Nepeta racemosa, Rosmariunus x lavandulaceus, Thymus funkii, Coridothymus capitatus, Origanum syriacum, Thymus cilicicus, Eucalyptus porosa, Laurus nobilis, Daucas carota, Eucalyptus leucoxylon, Teucrium micropodioides, Leonotis leonurus, Micromeria varia subsp. thymoides, Hyptis suaveolens, Plectranthus coleoides, Vitex agnus-castus, Calamintha nepeta, Micromeria myrtifolia, Mentha aquatic, Salvia dorisiana, Ocimum suave, Sideritis scardica, Plectranthus incanus, Mentha x piperita, Hyssopus officinalis subsp. aristatus, Rosmarinus x mendizabalii, Satureja subspicata subsp. librnica, Sideritis mugronesis, Eucalyoptus fasiculosa, Teucrium gnaphalodes, Dictamnus albus, Satureja cilicica, Monardia citriodora, Sideritis germanicolpitana, Zingiber officinale, Eucalyptus sparsa, Thymus longicaulis, Origanum vulgare var. gracile, Minthostachys mollis, Monardia didyma, Salvia sclarea, Eucalyptus melanophloia, Elsholtzia blanda, Eucalyptus desquamate, Teucrium pseudoscorodonia, Eucalyptus cuprea, Sideritis pauli, Eucalyptus lansdowneana, Teucrium salviastrum, Teucrium scorodonia, Elsholtzia eriostachya var. pusilla, Sideritis athoa, Aralia cordata, Eucalyptus intertexta, Teurcrium oxylepis subsp. oxylepis, Cleaonia lusitanica, Satureja cuneifolia, Eucalyptus largisparsa, Eucalyptus odorata, Teurcrium polium var. valentinum, Eucalyptus behriana, Eucalyptus populnea, Teurcrium oxylepis subsp. marianum, Origanum vulgare var. viride, Eucalyptus ochrophloia, Eucalyptus viridis, Teucrium asiaticum, Thymus zygis, Lonicera japonica, Achillea millefolium, Aesculus hippocastanum, Agastache rugosa, Alpinia galangal, Anethum graveolens, Angelica archangelica, Annona squamosal, Apium graveolens, Artemisia absinithium, Artemisia annua, Artemisia capillaris, Bidens pilosa, Boswellia sacra, Camellia sinensis, Carum carvi, Centella asiatica, Chamaemelum nobile, Chrysanthemum parthenium, Chrysanthemum x morifolium, Cinnamomum aromaticum, Cinnamomum camphora, Cinnamomum verum, Citrus limon, Citrus paradise, Citrus reticulate, Citrus sinensis, Coleus barbatus, Coriandrum sativum, Croton eluteria, Croton lechleri, Ellettaria cardamomum, Ephedra sinica, Eruca sativa, Eucalyptus albens, Eucalyptus angulosa, Eucalyptus astringens, Eucalyptus blakelyi, Eucalyptus bosistoana, Eucalyptus botryoides, Eucalyptus brassiana, Eucalyptus camaldulensis, Eucalyptus ceratocorys, Eucalyptus cladocalyx, Eucalyptus dealbata, Eucalyptus diversicolor, Eucalyptus dolichorhyncha, Eucalyptus erythrandra, Eucalyptus forrestiana, Eucalyptus globulus, Eucalyptus grandis, Eucalyptus incrassate, Eucalyptus maculata, Eucalyptus maiden, Eucalyptus melliodora, Eucalyptus moluccana, Eucalyptus occidentalis, Eucalyptus oviformis, Eucalyptus polyanthemos, Eucalyptus puncata, Eucalyptus siderophloia, Eucalyptus sideroxylon, Eucalyptus stoatei, Eucalyptus tereticornis, Eucalyptus tetraptera, Foeniculum vulgare, Hedychium flavum, Houttuynia cordata, Lantana camara, Leptospermum scoparium, Lindera benzoin, Magnolia denudate, Matricaria recutita, Malaleuca altemifolia, Melia azedarach, Mentha arvensis var. piperascens, Mentha pulegium, Mentha rotundifolia, Mentha spicata, Montanoa tomentosa, Murraya koenigii, Myrciaria dubia, Myristica fragrans, Myrrhis odorata, Nepeta cataria, Ocimum gratissimum, Panax ginseng, Pelargonium citrosum, Perilla frutescens, Petroselinum crispum, Pimenta dioica, Pimenta racemosa, Pimpinella anisum, Pinus strobus, Piper nigrum, Pistacia lentiscus, Populus tacamahacca, Psidium guajava, Ptychopetalum olacoides, Ravensara aromatic, Sambucus nigra, Vaccinium myrtillus, Sassafras albidum, Satureja hortensis, Stevia rebaudiana, Illicium verum, Gossypium sp., Tagetes filifolia, Tagetes lucida, Tagetes minuta, Tamarindus indica, Tanacetum parthenium, Teucrium polium, Trifolium pretense, Valeriana officinalis, Zea mays, Piper betel, Pycnanthemum tenuifolium, Thymus serpyllum, Pycanthemum setosum, Pycnanthemum pycnanthemoides, Pycnanthemum virginianum, Thymus orospedanus, Pycnanthemum clinopodioides, Pycnanthemum loomisii, Pilocarpus microphyllus, Hedeoma hispida, Lavandula x intermedia, Cymbopogon nardus, Pycnanthemum pilosum, Cuminum cyminum, Pycnanthemum verticillatum, Thymus capitatus, Pycnanthemum muticum, Lepechinia calycina, Aloysia citrodora, Dracocephalum thymiflora, Leonurus cardiac, Lepechinia schiediana, Scutellaria galericulata, Hedeoma pulegioides, Micromeria croatica, Pycanthemum californicum, Cunila origanoides, Pycnanthemum torreyi, Thymus mastichina, Lycopus europeus, Moldavica thymiflora, Juniperis communis, Satureja vulgaris, Elsholtzia polystachya, Lycopus virginicus, Scutellaria churchilliana, Pycnanthemum montanum, Agastache foeniculum, Agastache nepetoides, Carthamus tinctorius, Dracocephalum parviflora, Pycnanthemum beadle, Scutellaria parvula, Echinacea spp, Galeopsis tetrahit, Satureja douglasii, Balotta nigra, Ribes nigrum, Isanthus brachiatus, Moldavica parviflora, Elsholtzia cristata, Elsholtzia pilosa, Myrtus communis, Cordia verbenacea, Ferula galbaniflua, Commiphora gileadensis, Populus balsamifera, Citrus bergamia, Tanacetum annum, Abies balsamea, Ocimum basilicum ct linalool, Mentha citrate, Picea mariana, Malaleuca leucadendron var. cajuputi, Eriocephalus punctulatus, Cymbopogon winterianus, Pinus nigra laricio, Cupressus sempervirens, Psudotsuga menzies, Canarium luzonicum, Eucalyptus citriodora, Eucalypotus dives, Eucalyptus radiata, Agonis fragrans, Bowsellia carterii, Pelargonium roseum x asperum, Helichrysum bracteiferum, Helichrysum gymnocephalum, Helichrysum odoratissimum, Tsuga Canadensis, Malaleuca teretifolia, Citrus hystrix, Kunzea ambigua, Larix laricina, Lavendula angustifolia, Lavendula officinalis, Cymbopogon citradis, Cymbopogon citratus ct rhodinol, Citrus aurantifolia, Bursera delpechiana, Origanum marjorana, Litsea cubeba, Citrus aurantium var. amara, Malaleuca quinquenervia ct 1,8 cineole, Pinus resinosa, Cymbopogon martini var. motia, Pogostemom cablin, Citrus aurantium var. bigardia, Pinus edulis, Pinus ponderosa, Cinnamomum camphor act 1,8 cineole, Rhododendron anthopogon, Rose damascena, Rosa damascena/Pelargonium Roseum x asperum, Rosmarinus officinalis ct camphor, Rosmarinus officinalis ct verbenone, Aniba rosaeodora, Cinnamosma fragrans, Pinus sylvestris, Abies sibirica, Abies alba, Lavandula latifolia, Hypericum perforatum, Cinnamomum glaucescens, Cinnamomum tamala, Thymus zygis \, Thymus vulgaris ct linalool, Ocimum sanctum ct eugenol, Thymus vulgaris ct thymol, Curcuma longa, Picea glance, Zanthoxylum armatum, Cananga odorata, Ocimum mircanthum, Ocimum selloi, Citrus junos, and all Plantae taxa thereof, including, life, domain, kingdom, phylum, class, order, family, genus, species, super-species, sub-species, varieties, hybrids and chemotypes, phenotypes and genotypes, whether naturally occurring or genetically modified. Preferred plant sources of beta-caryophyllene include at least about 20% beta-caryophyllene, such as, at least about 25%, 30%, 35%, 40%, 45% or 50% beta-caryophyllene. Copaiba/Copaifera sp., cloves, and cinnamon, as well as herbs like oregano, basil, hops, C. sativa/indica and rosemary, are known to exhibit high concentrations of caryophyllene.

Functionally equivalent derivatives, analogues or salts of beta-caryophyllene, which are pharmaceutically acceptable, may replace beta-caryophyllene in the present composition. The term “functionally equivalent” as used with respect to derivatives, analogues and salts of beta-caryophyllene, refers to compounds which possess the activity or function of beta-caryophyllene, at least in part, to treat pain and/or inflammation.

Functionally equivalent derivatives or analogues, including structural and functional analogues, of beta-caryophyllene include compounds derived from beta-caryophyllene or a precursor thereof, including isomers thereof. Examples of functionally equivalent derivatives or analogues include, but are not limited to, alpha-humulene, 9-epi-(E)-Caryophyllene, [−]-Caryophyllene oxide or (−)-Epoxycaryophyllene, (li?,4i?,6?,10S)-9-Methylene-4,12,12-trimethyl-5-oxatricyclo[8.2.0.0]Caryophyllene, 9-epi-Caryophyllene or (E)-Caryophyllene, epi-, cis-Caryophyllene, (+)(E)-Caryophyllene or 2-epi-(E)-β-Caryophyllene, and Isocaryophyllene (or (Z)-Caryophyllene or β-cis-Caryophyllene or (Z)-Caryophyllene or Bicyclo(7.2.0)undec-4-ene, 4,1 1,1 1-trimethyl-8-methylene-, (1R,4Z,9S)- or cis-Caryophyllene or γ-Caryophyllene or [1R-(1R*,4Z,9S*)]-4,1 1, 1 1-trimethyl-8-methylenebicyclo[7.2.0]undec-4-ene).

Functionally equivalent salts which are pharmaceutically acceptable salts of beta-caryophyllene are also encompassed herein for use to treat inflammation. The term “salts” refers to salts or esters of beta-caryophyllene that retain the desired biological activity of the parent compound to treat pain and/or inflammation, at least in part. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Although various natural sources of beta-caryophyllene exist which may be incorporated into the present composition, it will be appreciated that beta-caryophyllene for use in the present composition may also be synthetically derived.

### CBD

It should be appreciated that in the context of the present invention the terms “cannabidiol compound”, “cannabidiol” or “CBD” (which may be used interchangeably unless the context clearly dictates otherwise) refer to any natural, semi-synthetic or synthetic cannabinoid compound.

CBD, unless a particular other stereoisomer or stereoisomers are specified, includes the compound “Δ²-cannabidiol.” These compounds are: (1) Δ⁵-cannabidiol (2-(6-isopropenyl-3-methyl-5-cyclohexen-1-yl)-5-pentyl-1,3-benzenediol); (2) Δ⁴-cannabidiol (2-(6-isopropenyl-3-methyl-4-cyclohexen-1-yl)-5-pentyl-1,3-benzenediol); (3) Δ³-cannabidiol (2-(6-isopropenyl-3-methyl-3-cyclohexen-1-yl)-5-pentyl-1,3-benzenediol); (4) Δ^{3,7}-cannabidiol (2-(6-isopropenyl-3-methylenecyclohex-1-yl)-5-pentyl-1,3-benzenediol), (5) Δ²-cannabidiol (2-(6-isopropenyl-3-methyl-2-cyclohexen-1-yl)-5-pentyl-1,3-benzenediol); (6) Δ¹-cannabidiol (2-(6-isopropenyl-3-methyl-1-cyclohexen-1-yl)-5-pentyl-1,3-benzenediol); and (7) Δ⁶-cannabidiol (2-(6-isopropenyl-3-methyl-6-cyclohexen-1-yl)-5-pentyl-1,3-benzenediol).

CBD may include cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannahivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

According to one embodiment, the CBD compound comprises the following general Formula (I):

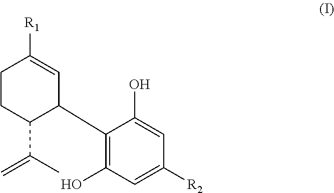

Wherein R1 is an alkyl; and

R2 is selected from a straight or branched alkyl having 5 to 12 carbon atoms; an —OR3 group, wherein R3 is a straight or branched alkyl having 5 to 9 carbon atoms or a straight or branched alkyl substituted at the terminal carbon atom by a phenyl group; or a —(CH₂)n-O-alkyl group, wherein n is an integer from 1 to 7 and the alkyl group has 1 to 5 carbons.

In one embodiment, R1 is CH₃ and R₂ is a straight alkyl having 5 carbon atoms (i.e. —C₅H₁₁).

In another embodiment, the CBD compound is cannabidiol. CBD, the molecular formula of C₂₁H₃₀O₂, as depicted in the following formula (II):

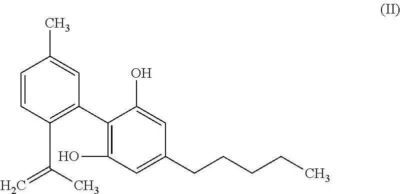 [SMILES: C=C(C)c1ccc(C)cc1-c1c(O)cc(CCCCC)cc1O]

The cannabidiol of formula (II) may be a natural cannabidiol obtainable by extraction from a plant member of the genus Cannabis or any preparations of Cannabis (e.g., processed plant material). According to one embodiment, the natural cannabidiol may be extracted from Cannabis sativa or one of its preparations, e.g., marijuana, hashish, etc. According to one embodiment, the natural cannabidiol can be extracted from Cannabis using methods such as described, for example, in U.S. Pat. No. 6,403,123, and in Gaoni and Mechoulam [J Chem Soc 93:217-224 (1971)], both incorporated herein by reference.

The cannabidiol may also be a synthetic cannabidiol or a derivative thereof which can be generated using methods such as those described, for example and without being limited thereto, in US 2003/166727, and incorporated herein by reference.

In some embodiments, the composition comprises a CBD derivative. The term “CBD derivative” as used herein means a CBD derivative having an anti-inflammatory effect, or an analgesic effect, or having an ameliorating effect on inflammatory disease, disorder or conditions; or alternatively, a CBD derivative that binds to CB(1) and/or CB(2) cannabinoid receptors.

In some embodiments, a CBD derivative is selected from (−)-7-hydroxy-CBD, which is known from WO 2015/198077 to reduce triglyceride levels and treat fatty liver; (−)-CBD-7-oic acid, which is known from Haj 2015 to have an anti-inflammatory effect; and the dimethylheptvl (DMH) homolog of CBD, which is known to have an anti-inflammatory effect (Ben-Shabat 2006; Juknat 2016), and the corresponding compounds in the enantiomeric (+)-CBD series.

In some embodiments, a CBD derivative is characterized by a structure, wherein at least one of the hydroxyl substituent groups is converted to a stable form thereof. In some embodiments, a CBD derivative is cannabinol comprising a quinone ring. In one embodiment, a CBD derivative is an endocannabinoid derivative. In another embodiment, the pentyl group on the phenyl ring of the CBD is replaced with any straight-chain or branched alkyl group selected from (C1-C18)alkyl, optionally substituted.

In some embodiments the CBD is prepared from a cannabis extract. In some embodiments the term “CBD or a derivative thereof” refers to between 80% and 99% pure CBD. In some embodiments the term “CBD or a derivative thereof refers to between 90% and 99% pure CBD. In some embodiments the term “CBD or a derivative thereof” refers to between 93% and 99% pure CBD. In some embodiments the term “CBD or a derivative thereof refers to between 95% and 99% pure CBD. In some embodiments the term “CBD or a derivative thereof refers to between 95% and 97% pure CBD. In some embodiments the term “CBD or a derivative thereof” refers to about 97% pure CBD. All % hereinabove are weight %.

In some embodiments, the CBD or a derivative thereof is substantially and/or essentially devoid of tetrahydrocannabinol (THC). In some embodiments, a composition of the invention, as described herein, is substantially and/or essentially devoid of THC. In one embodiment, substantially and/or essentially devoid of THC means less than 10%» by weight THC. In one embodiment, substantially and/or essentially devoid of THC is less than 7% by weight THC. In one embodiment, substantially and/or essentially devoid of THC is less than 5% by weight THC. In one embodiment, substantially and/or essentially devoid of THC is less than 3% by weight THC. In one embodiment, substantially and/or essentially devoid of THC is less than 1% by weight THC. In one embodiment, substantially and/or essentially devoid of THC is less than 0.5% by weight THC. In one embodiment, substantially and/or essentially devoid of THC is less than 0.3% by weight THC. In one embodiment, substantially and/or essentially devoid of THC is less than 0.1% by weight THC.

In some embodiments, the CBD is synthetically prepared.

### Tributyrin

Tributyrin is a triglyceride and a butyrate ester that may be obtained by formal acylation of the three hydroxy groups of glycerol by butyric acid. As used herein, a derivative of tributyrin includes beta hydroxybutyrate, monobutyrin, dibutyrin, triacetin, tripropionate, glyceryl monoacetate, glyceryl diacetate, acetoacetate, a butyrate mono-ester, prodrug of butyric acid, a butyrate di-ester, and any derivatives thereof.

A pharmaceutically effective amount of butyrate may be administered via triglyceride tributyrin (e.g., glyceryl tributyrate or tributyrin). The butyrate via triglyceride tributyrin may be administered separately and/or in conjunction with one or more of the other described compounds (e.g., beta-hydroxybutyrate, fatty acids and/or esters, etc.). For example, up to approximately 200 mg/kg of the individual may be administered (e.g., up to 3 times daily). Administration of the tributyrin may allow a delayed release of butyrate to the body as the tributyrin is processed by the body of the individual. The tributyrin may be unencapsulated and/or encapsulated (e.g., microencapsulated). Tributyrin may be organic and non-organic salt cations.

Tributyrin (butyric acid prodrug)/butyric acid equivalents include Mineral salts selected from the group consisting of: butyrates, Sodium Butyrate, Magnesium butyrate, Potassium butyrate, Calcium butyrate, and organic and non-organic salt cations. Tributyrin (butyric acid prodrug)/butyric acid equivalents include Esters-butanoates selected from group consisting of: Butyl butyrate, Butyryl-CoA, Cellulose acetate butyrate, Estradiol benzoate butyrate, Ethyl butyrate, Methyl butyrate, and Pentyl butyrate. Tributyrin (butyric acid prodrug)/butyric acid equivalents include Amino Butyrate esters, Tripropionin.

### Retinyl Palmitate Vit-A

Retinyl palmitate, an ester form of retinoic acid (Vitamin A), and precursor of 9-cis retinyl palmitate, or derivatives of Vitamin A include, but not limited to, retinol palmitate, retinyl palmitate, retinol acetate, beta-carotene or combinations thereof. Alternatively, the retinoid may be all-trans-, 9-cis-, 11-cis retinoic acid. Retinyl palmitate may include trans-Retinyl palmitate, cis-retinyl palmitate, 2-retinyl palmitate, 3-retinyl palmitate, or combinations thereof. Vitamin A or retinyl palmitate may also include retinoid receptor agonist including AM-580, BMS641, BMS961, CD666, TTNPB, ATRA, Ro25-7386, methoprene acid or combinations thereof. Natural and non-natural derivative of retinoids or retinyl palmitate are also contemplated. The skilled artisan may determine what derivatives of retinoids or retinyl palmitate have similar activity to naturally occurring compounds. Other agonists that may activate the receptors RAR/RXRα, β or γ are also contemplated by the present invention. All retinoic acid/alpha/beta-carotene congeners & retinyl esters that are ligands & signal via RXR receptors are contemplated herein.

Vitamin A equivalents include Beta/alpha carotene, Beta-cryotoxanthin, Lutein, Zeaxanthin, Lycopene, Tazarotene (brand name: Tazorac), Adapalene, and Retinyl esters selected from the group consisting of: Palmitic acid, Oleic acid, Stearic acid, and Linoleic acid.

### DESCRIPTION

Multicomponent therapeutics, in which two or more agents interact simultaneously with multiple targets is a rational and efficient form of therapy designed to control complex diseases. One of the advantages of multicomponent therapeutics is the potential synergistic effect of the combination, e.g. an effect which is greater than the sum of the expected individual effects. A multimodal therapeutic approach is best suited to target the complex mechanisms leading to the inflammation and inflammatory related diseases and conditions. Multi-target drugs may overcome system robustness and result in reduced side-effects and reduced toxicity that may be associated with high doses of single drugs.

Generally speaking, the method and composition comprises use of at least one short fatty acid, such as butyrate or butyric acid in form of tributyrin, used in conjunction with one cannabinoid, such as cannabidiol or CBD, and with at least one terpene, such as beta-caryophyllene (BCP), to treat general inflammation or an inflammatory condition.

An inflammatory condition may include, but is not limited to: I) Metabolic health/insulin resistance/metabolic syndrome/glycemic-glucose uptake & management (skeletal muscle, liver, adipose, dermis/epidermis/skin, gastrointestinal/gut, fibro-connective tissues and brain/CNS); II) Conditions that require Immuno-modulation/Optimal Inflammation Response/Active Resolution/Anti-Inflammatory therapeutics; III) Mitigate Neuroinflammation/Neurodegenerative changes (i.e., optimize neuro-restoration/neuroprotection); IV) non-alcoholic steatohepatitis (NASH) and/or non-alcoholic fatty liver disease (NAFLD) NASH/NAFLD (Fatty Liver) & Metabolic Associated Hepatic Dysfunction; V) Mitigate Pulmonary Fibrosis & Optimize pulmonary function; and VI) Renal/Nephroprotective.

For the purpose of this disclosure, inflammatory diseases and conditions include any disease that is associated with inflammation, such as for example viral infections or bacterial infections, cardiovascular diseases, autoimmune diseases, eye diseases, including (but not limited to) dry eyes, conjunctivitis, uveitis, pink eyes, keratoconjunctivitis of any origin (including viral, bacterial and allergic); mucositis such as chemotherapy and radiation induced mucositis or gastro intestinal inflammation; inflammatory bowel diseases, ulcerative colitis and Crohn's disease, inflammatory gastric and intestinal ulcers, skin inflammation and skin inflammation associated with dry skin, atopic dermatitis, psoriasis and similar skin diseases; ear, nose and throat infections and non-infective inflammatory conditions; vaginal infections and other vaginal inflammatory conditions; anal or rectal inflammation; inflammation of any tissue or organ that results from physical, metabolic or chemical insult, from heat, irradiation, auto immune disease, oxidation stress or chemotherapy. Many neurological and central nervous system (CNS) diseases such as multiple sclerosis and Alzheimer disease are related to chronic inflammatory condition. Atherosclerosis is also considered to possess autoimmune and chronic inflammatory aspects in its pathogenesis. Tumor microenvironment is an inflammatory state which is an immune suppressive condition, thus hindering the body immune response to attack the cancer.

Biomarkers of the inflammatory condition may include, but are not limited to: Cellular AOx capacity (AML-12 liver cells and C2C12 muscle cells), Superoxide dismutase 2 protein (SOD2) (AML-12 liver cells and C2C12 muscle cells), Peroxisome proliferator-activated receptors gamma (PPAR-γ) DNA binding activity (AML-12 liver cells and C2C12 muscle cells), Inflammatory signaling (pro-caspase-1) (AML-12 liver cells and C2C12 muscle cells), Inflammatory signaling (phosphorylated NF-kB) (AML-12 liver cells and C2C12 muscle cells), Cellular glucose levels (AML-12 liver cells and C2C12 muscle cells).

In some embodiments, a conjugated formulation provided herein may include 0.1 mg/ml to 40 mg/ml of beta-caryophyllene. Conjugated is not limited to using chemically “bonded” compositions of these bioactives. In some embodiments, the conjugated formulation may be admixtures of the bioactives. For example, a conjugated formulation provided herein may comprise 0.1 mg/ml to 30 mg/ml, 0.5 mg/ml to 30 mg/ml, 1 mg/ml to 30 mg/ml, 0.1 mg/ml to 25 mg/ml, 0.5 mg/ml to 25 mg/ml, 1 mg/ml to 25 mg/ml, 0.1 mg/ml to 20 mg/ml, 0.5 mg/ml to 20 mg/ml, 1 mg/ml to 20 mg/ml, 0.1 mg/ml to 15 mg/ml, 0.5 mg/ml to 15 mg/ml, 1 mg/ml to 15 mg/ml, 0.1 mg/ml to 10 mg/ml, 0.5 mg/ml to 10 mg/ml, 1 mg/ml to 10 mg/ml, 0.1 mg/ml to 5 mg/ml, 0.5 mg/ml to 5 mg/ml, 1 mg/ml to 5 mg/ml, 0.1 mg/ml to 2 mg/ml, 0.5 mg/ml to 2 mg/ml, 1 mg/ml to 2 mg/ml, 2 mg/ml to 40 mg/ml, 2 mg/ml to 30 mg/ml, 2 mg/ml to 25 mg/ml, 2 mg/ml to 20 mg/ml, 2 mg/ml to 15 mg/ml, 2 mg/ml to 10 mg/ml, 2 mg/ml to 5 mg/ml, 5 mg/ml to 40 mg/ml, 5 mg/ml to 30 mg/ml, 5 mg/ml to 25 mg/ml, 5 mg/ml to 20 mg/ml, 5 mg/ml to 15 mg/ml, 5 mg/ml to 10 mg/ml, 10 mg/ml to 40 mg/ml, 10 mg/ml to 30 mg/ml, 10 mg/ml to 25 mg/ml, 10 mg/ml to 20 mg/ml, 10 mg/ml to 15 mg/ml, 15 mg/ml to 40 mg/ml, 15 mg/ml to 30 mg/ml, 15 mg/ml to 25 mg/ml, 15 mg/ml to 20 mg/ml, 20 mg/ml to 40 mg/ml, 20 mg/ml to 30 mg/ml, 20 mg/ml to 25 mg/ml, 25 mg/ml to 40 mg/ml, 25 mg/ml to 30 mg/ml, 30 mg/ml to 40 mg/ml, or between 5 mg/ml and 125 mg/ml of beta-caryophyllene.

In some embodiments, a conjugated formulation provided herein may comprise at least 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or between 0.45 mg/ml to 4.5 mg/ml of Vitamin A.

In some embodiments, a conjugated formulation provided herein may comprise 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, or between 50 mg/ml and 5000 mg/ml of tributryin.

In some embodiments, a conjugated formulation provided herein may comprise 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or between 400 mg/ml and 4000 mg/ml of CBD.

In one embodiment, Vitamin A, beta-caryophyllene, and tributryin may be at a ratio of about 0.45 to about 4.5 mg of Vitamin A to: about 5 mg to about 125 mg of beta-caryophyllene to about 50 mg to about 5000 mg of tributryin.

In another aspect, the present invention encompasses a method for treating an inflammatory condition or disorder in a mammalian subject (preferably, but not exclusively, a mammalian subject) in need of such treatment, wherein said method comprises the administration (systemically, topically or by a combination of routes) of a composition of the present invention.

The present invention further provides a conjugated formulation for use as medicament or other therapeutic entity (such as ‘herbal remedy’, ‘food supplement’ and the like) in the treatment of an inflammatory condition. In one embodiment of this aspect, said composition is provided for use as a medicament or other therapeutic entity in the treatment of a systemic or topical or mucosal inflammatory disorder.

The present invention further provides the use of a conjugated formulation as disclosed herein for the preparation of a medicament. In some embodiments, this aspect of the invention relates to the use of a conjugated formulation as disclosed herein in the preparation of a medicament for use in the treatment of an inflammatory condition or disorder.

The present invention unexpectedly found that 1-beta-caryophyllene plus cannabidiol and tributyrin; 2-Retinyl palmitate (Vit A) plus beta-caryophyllene and cannabidiol; 3-Retinyl palmitate (Vit A) plus beta-caryophyllene controlled inflammation markers, apoptosis as well as enhance cellular antioxidant activity and a synergistic reduction of oxidative stress using a conjugated formulation comprising a combination of beta-caryophyllene, CBD, and tributryin as well as the conjugated formulation of Vitamin A, beta-caryophyllene, and CBD, as well as the conjugated formulation of Vitamin A and beta-caryophyllene. Surprisingly, the efficacy of this combination in inhibiting inflammatory mediators is comparable to the efficacy of DMSO, as will be shown in the experimental results presented hereinbelow.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, toffee, chocolate or cookie. In certain embodiments, the tablet or pellets are an immediate release or slow or controlled release dosage forms. In certain embodiment the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the cannabinoids at the distal intestine and may also release part of the cannabinoids in the intestines for systemic absorption and part of the cannabinoids at the colon for local colonic pharmacological effect.

In certain embodiments, the conjugated formulation is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form.

In certain embodiments, any one of the compositions described above, or any one of the dosage forms described above, is for use in a method of treating inflammation symptoms or disorders.

Preferred dosage forms include, but are not limited to, any liquid or semi solid or solid dosage form. The composition may be formulated in a medicament by preparing a topical or mucosal or oral delivery system. The topical delivery system may be in form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the composition further comprises one or more excipients selected from the group consisting of solvents, stabilizers, suspending agents, emulsifiers, release modifying, targeting and viscosity agents and combinations thereof.

In some embodiments, the composition of the present invention is formulated as a dosage form selected from the group consisting of a liquid, a suspension, an emulsion, a foam, a spray, a liposome, a semi-solid, a cream, an ointment, a patch, a particulate formulation, a granulate, a micro-particulate formulation, a nano-particulate formulation, a solid dosage form, a tablet, a capsule, an orally-disintegrable capsule, a mouth wash and an adhesive buccal tablet.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Testing the Effects of Different Ingredients on Biomarker Responses in Muscle and Liver Cells In Vitro

### Materials and Methods

Testing the effects of different ingredients on biomarker responses in muscle and liver cells in vitro.

### Treatments and Cells

This example shows the treatments with one of five compositions: 1) Control or “vehicle” (DMSO only); 2) CBD (˜95 ng/ml); 3) Vitamin A, or “VitA” (˜1.2 μM); 4) Tributyrin (˜10 μM); 5) Beta-caryophyllene (˜100 ng/ml)

There were two different treatment experiments including: Cells treated with compounds 1-5 above for 3 hours; Cells treated with compounds 1-5 above for 12 hours with a concomitant TNF-alpha treatment (15 ng/mL); Cell treatments were performed using C2C12 myotubes (muscle cells), and AML-12 cells (liver cells).

There are also combination formulations, where the following combination formulations were tested: 6) Control or “vehicle” (DMSO only); 7) Vitamin A, or “VitA” (1.2 μM)+Beta-caryophyllene (100 ng/mL)+Tributyrin (10 μM); 8) Vitamin A, or “VitA” (1.2 μM)+Beta-caryophyllene (100 ng/mL)+CBD (95 ng/ml); 9) Beta-caryophyllene (100 ng/mL)+CBD (95 ng/mL)+Tributyrin (10 μM); 10) Vitamin A, or “VitA” (1.2 μM)+Beta-caryophyllene (100 ng/mL).

As with single ingredients, two different treatment experiments occurred with these combos including: Cells treated with compounds 1-5 above for 3 hours. Cells treated with compounds 1-5 above for 12 hours with a concomitant TNF-alpha treatment (15 ng/ml)

### General Culture Methods

All cells were grown to confluence using cell-specific media and additives.

The day of treatment, culture media was spiked with the appropriate ingredients (these are termed “treatment media”)

After either the 3-h or 12-h treatments described above, treatment media was removed, cells were rinsed with 1× phosphate-buffered saline, and general cell lysis buffer (Tris-based with detergent) was used to lyse cells

Cells were scraped off the plate with a rubber policeman in the lysis buffer, the slurry was placed in 1.7 mL tubes, and lysates were frozen at −80° C. until the respective assays discussed below.

There are n=5-6 replicates per treatment condition.

### Molecular Markers Assessed

Total antioxidant capacity was assessed using a Cu2+ ion is conversion assay. Notably, all data was normalized to total protein loaded in the assay.

iNOS activity assays were performed using an enzymatic kit. Notably, all data was normalized to total protein loaded in the assay. The iNOS assay yielded low-to-no signal for most treatments.

PPARγ DNA binding activity was assessed using a plate that was pre-coated with a PPAR-gamma consensus binding sequence. Thereafter, antibody-based methods were used to assess the amount of bound transcription factor, and data were normalized to total protein loaded in the assay.

Cellular glucose was assayed using a colorimetric glucose assay kit that implemented the glucose oxidase enzyme reaction.

SOD2, SOD1, phosphorylated p65/NF-κB, pro-caspase 1, and NLRP3 were assayed using Western blotting methods. Notably, NLRP3 yielded low-to-no signal for most treatments.

### Result and Discussion

All examples showed both the significance values as well as the comparison in relation to the synergy between the ingredients in liver cell and muscle cell. The results refer to the ingredients tested in isolation and ingredients together or conjugated formulation.

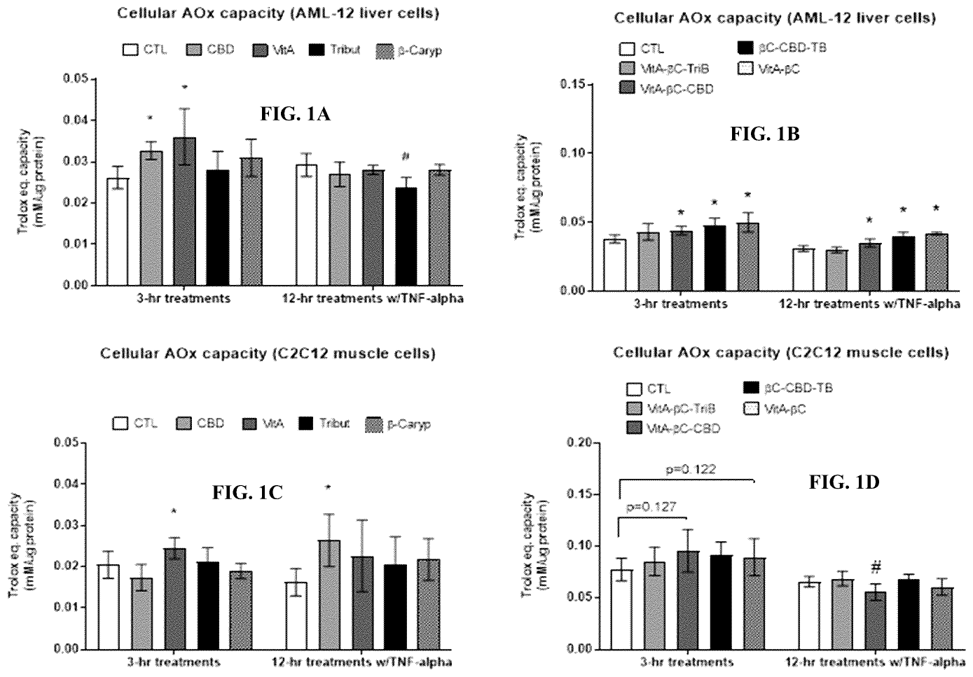

As shown in FIG. 1A, in isolation treatment the result showed an antioxidant capacity of vitamin A in liver cells in relation to the other ingredients only in a period of 3 hours of treatment. The CBD also expressed results with significance value in liver cells in the same period, but less expressive. When the cells were submitted to 12 h of treatment with TNF-alpha, there was no significance value in any of the ingredients individually.

As shown in FIG. 1C, in muscle cells, vitamin A also expressed significance values in relation to the other ingredients in the 3-hour treatment. Notably, CBD expressed significance value when these cells were exposed to 12 h treatment with TNF-alpha.

In treatments with combined formulations, the combinations of Vit A, beta-caryophyllene (BCP), and CBD, BCP, CBD and tributyrin, and Vit A and BCP expressed results with significance value in liver cells in both the 3 h and 12 h treatment with TNF-alpha, as shown in FIG. 1B. These results suggest that these conjugated formulas are treatments for oxidative stress, where tissues have little capacity to remove free radicals from disease.

As shown in FIG. 1D, in muscle cells there was no significance value in any of the groups in any of the treatments 3 h. On 12 h with TNF-alpha the group with Vit A, BCP and CBD showed a lower activity on cellular antioxidant capacity when compared to the control group.

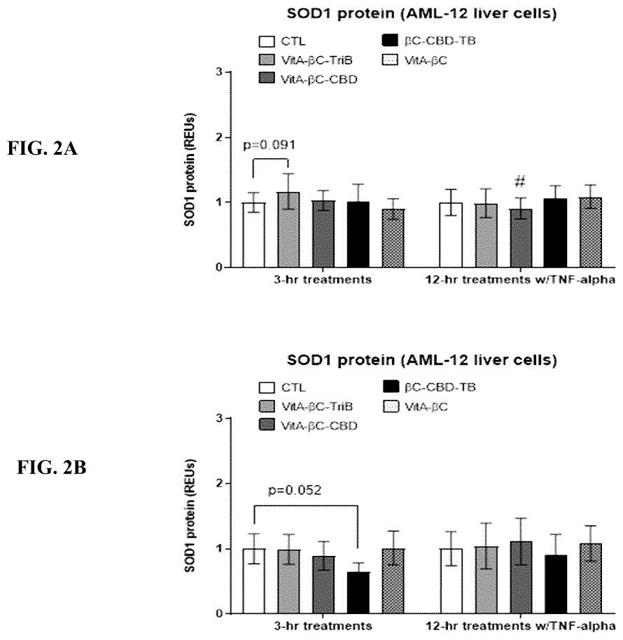

As shown in FIGS. 2A-2B, there were no results with significance value in both groups as well as in the two treatments 3 h and 12 h with TNF-alpha. Only the group with Vit A, BCP and CBD showed a lower activity on SOD1 when compared to the control group on liver cells.

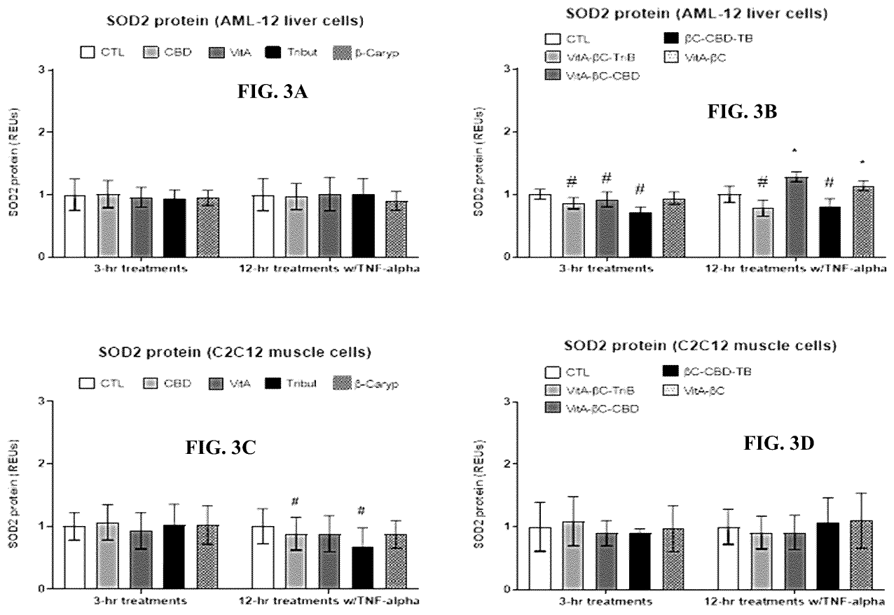

As shown in FIG. 3B, in liver cells, three conjugated formulas, among them Vit. A, BCP and tributyrin, Vit A, BCP and CBD, and BCP, CBD and tributyrin showed significance values less than control in SOD2 protein levels in the 3 h treatment. As shown in FIG. 3B, in the 12 h treatment with TNF-alpha, two combinations showed significance value greater than control in SOD2 activity compared to the control, among them Vit A, BCP and CBD, and Vit A and BCP, the first being the one that showed the best result in this group, which makes this combination treatments where oxidative stress predominates, such as in cases of physiological changes in hepatocytes.

As shown in FIG. 3D, in muscle cells, although there was no result with significant value in the combinations of ingredients in any of the treatments, some isolated ingredients showed significance value less the control, including CBD and tributyrin, as shown in FIG. 3C.

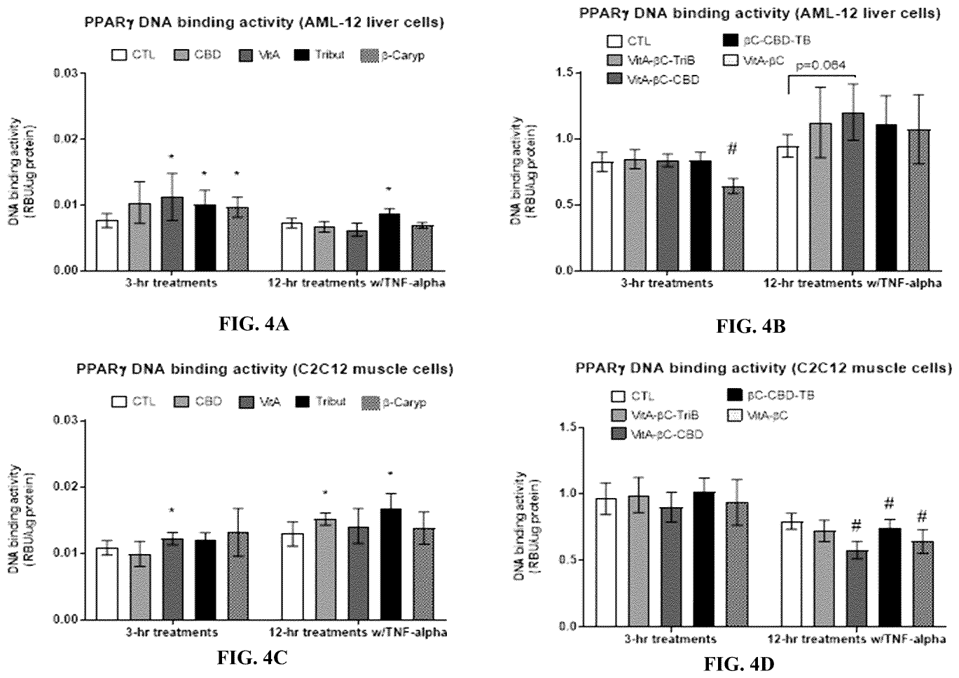

As shown in the FIG. 4A, in liver cells, the ingredients in isolation in the 3 h treatment to Vit. A, tributyrin and BCP showed significance values greater than control group in the binding activity of PPAR-gamma. However, in the treatment of 12 with TNF-alpha, only tributyrin presented results with a significance value greater than control group.

As shown in FIG. 4B, in the 3 h treatment the group with Vit. A and BCP showed a result less than control group, while the other groups did not show any result in relation to the significance values. On the other hand, although the conjugate groups had better results than the controls, none of them showed a significant value in the treatment of 12 with TNF-alpha.

As shown in FIG. 4C, in muscle cells only Vit. A presented a significance value greater than control group in the 3 h treatment. In the 12-hour treatment with TNF-alpha, CBD and tributyrin stood out, demonstrating values of greater significance than the control. As shown in FIG. 4D, in the 12 h treatment with TNF-alpha the Vit. A, BCP and CBD, BCP, CBD and tributytin, and Vit A and BCP showed significance less than control group in relation to PPAR-gamma activity.

The results presented with the ingredients conjugated in muscle tissue in the 12-hour treatment with TNF-alpha may be explained by the fact that some ingredients compete for the PPAR-gamma receptor in relation to the receptor's agonist activity, thus decreasing its activity in this type of tissue. A more specific study may be needed to clearly show why this decrease in PPAR-gamma activity occurs when the ingredients are used in combination.

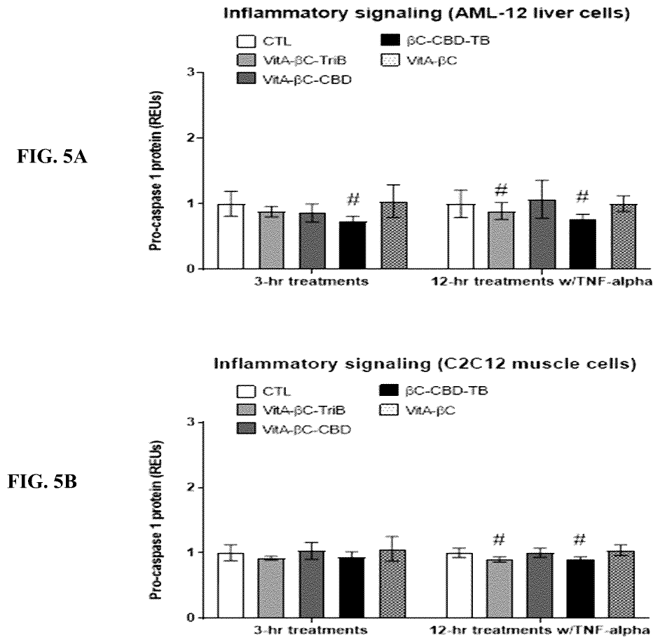

As shown in FIG. 5A, in liver cells, both in the 3-hour treatment and in the 12-hour treatment with TNF-alpha there was a decrease in inflammatory signaling with the conjugated formula BCP, CBD and tributyrin. This conjugated formula showed significance values less than control group, demonstrating great capacity to protect the liver tissue from apoptosis when it is exposed to inflammatory processes. The results over pro-caspase 1 showed that this formula can control the apoptotic mechanisms protecting the hepatocytes even when they are submitted to inflammatory processes.

As shown in FIG. 5A, the formula conjugated with Vit A, BCP and tributyrin also showed significance values less than control, but only in 12 h treatment with TNF-alpha, nonetheless with lower results than the formula conjugated with BCP, CBD and tributyrin.

As shown in FIG. 5B, in muscle tissue both formulas also showed significance values less than control group, but only in the 12-hour treatment with TNF-alpha.

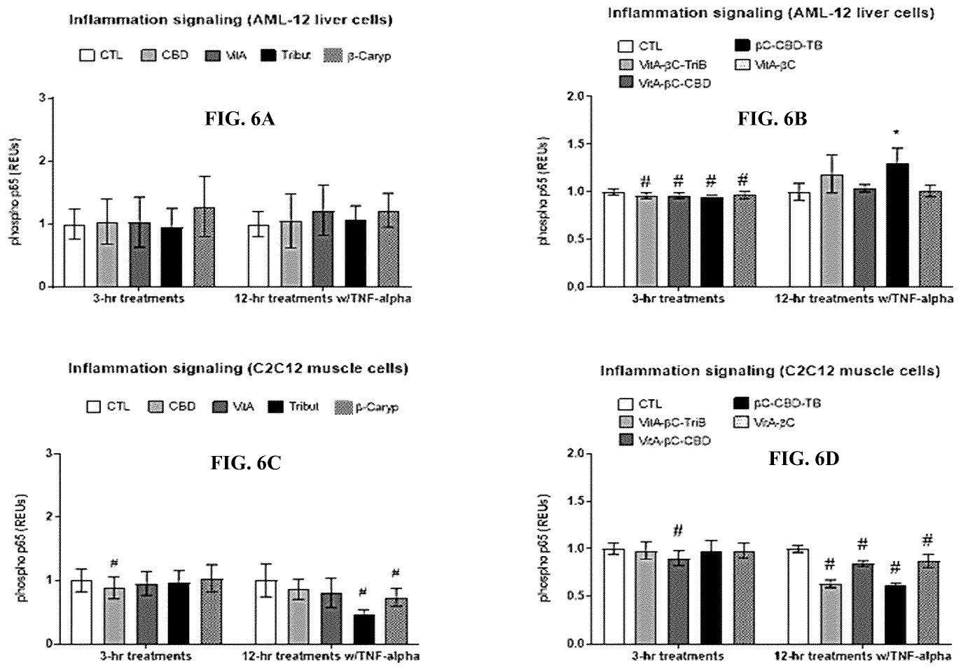

As shown in FIGS. 6B and 6D, the results in the treatments with the conjugated formulas are shown for inflammatory signaling and NF-kB phosphorylation. Although the treatments with the isolated ingredients did not present any significant value when compared to the control group, as shown in FIG. 6A, in the 3 h treatment all conjugated formulas showed a decrease in NF-kB phosphorylation when compared to the control group, as shown in FIG. 6B. However, in the 12 h treatment with TNF-alpha, the formula BCP, CBD and tributyrin, which had shown the best result in the 3 h treatment, significantly increased NF-kB phosphorylation.

As shown in FIG. 6D, in muscle tissue, the results were notable in all conjugated formulas in the 12 h treatment with TNF-alpha, mainly with Vit A, BCP and tributyrin, and BCP, CBD and tributyrin which showed the best results. In the 3 h treatment, only the Vit A, BCP and tributyrin showed significant values in decreasing NF-kB phosphorylation.

FIG. 6C shown that some ingredients in isolated form showed significant values in reducing NF-kB phosphorylation, including BCP and especially tributyrin in the 12-hour treatment with TNF-alpha. In the 3-hour treatment, CBD stood out, being the only one with significant value among the ingredients.

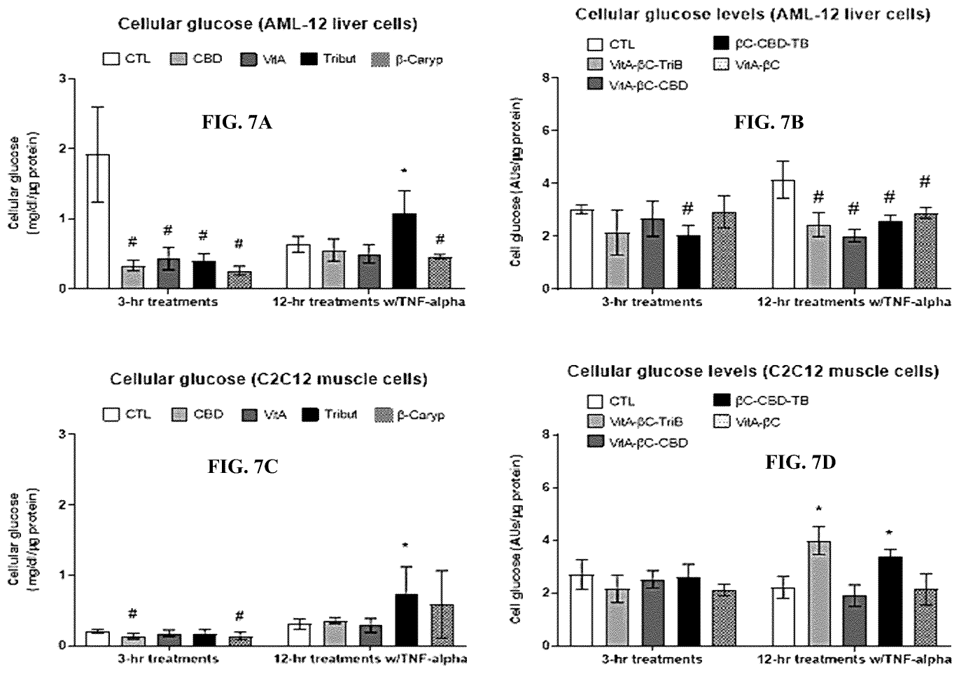

As shown in FIG. 7A, tributyrin increases cell glucose in liver cells in the 12-hour treatment with TNF-alpha. However, as shown in FIG. 7B, all conjugated formulas decreased cell glucose in the in the 12-hour treatment with TNF-alpha. As shown in FIG. 7C, tributyrin increases cell glucose in muscle cells in the 12-hour treatment with TNF-alpha. However, as shown in FIG. 7D, VitA_βC_TriB and βC_CBD_TriB increases cell glucose in muscle cells in the 12-hour treatment with TNF-alpha.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating an inflammatory condition comprising:
a. providing a composition comprising Vitamin A, beta-caryophyllene (BCP), and butyrate or butyric acid in form of tributyrin; and
b. administering an effective amount of the composition to the subject to act on the subject's inflammatory condition.

2. The method of claim 1, further comprising significantly increasing the cellular antioxidant capacity of the subject.

3. The method of claim 1, further comprising significantly increasing the Superoxide dismutase 2 protein (SOD2) activity of the subject.

4. The method of claim 1, further comprising significantly increasing the binding activity of PPAR-gamma in the subject.

5. The method of claim 1, further comprising significantly decreasing the inflammatory signaling in the subject.

6. The method of claim 1, further comprising significantly decreasing the NF-kB phosphorylation in the subject.

7. The method of claim 1, wherein the composition is a dietary supplement.

8. The method of claim 1, wherein the inflammatory condition is selected from the group consisting of: Metabolic health/Insulin Resistance/Metabolic Syndrome/Pre-diabetes/Disturbed glycemic-glucose uptake & management of multiple tissues and organ systems (including skeletal muscle, liver, adipose, skin/dermis/epidermis, gastrointestinal mucosal/gut, renal/kidney and brain/CNS), Dysfunctional Immuno-modulation/Perturbed Inflammation Response and Resolution/Anti-Inflammatory, Neuroinflammation/Neurodegenerative conditions, non-alcoholic steatohepatitis (NASH) and/or non-alcoholic fatty liver disease (NAFLD) (Fatty Liver) & Metabolic Associated Hepatic Dysfunction, Pulmonary Fibrosis & pulmonary dysfunction, and Renal dysfunction that require Nephroprotective measures.

9. A method of treating an inflammatory condition comprising:
a. providing a composition comprising butyrate or butyric acid in form of tributyrin, cannabinoid, and beta-caryophyllene (BCP); and
b. administering an effective amount of the composition to the subject to act on the subject's inflammatory condition.

10. The method of claim 9, further comprising significantly increasing the cellular antioxidant capacity of the subject.

11. The method of claim 9, further comprising significantly increasing the Superoxide dismutase 2 protein (SOD2) activity of the subject.

12. The method of claim 9, further comprising significantly increasing the binding activity of PPAR-gamma in the subject.

13. The method of claim 9, further comprising significantly decreasing the inflammatory signaling in the subject.

14. The method of claim 9, further comprising significantly decreasing the NF-kB phosphorylation in the subject.

15. The method of claim 9, wherein the composition is a dietary supplement.

16. The method of claim 9, wherein the inflammatory condition is selected from the group consisting of: Metabolic health/Insulin Resistance/Metabolic Syndrome/Pre-diabetes/Disturbed glycemic-glucose uptake & management of multiple tissues and organ systems (including skeletal muscle, liver, adipose, skin/dermis/epidermis, gastrointestinal mucosal/gut, renal/kidney and brain/CNS), Dysfunctional Immuno-modulation/Perturbed Inflammation Response and Resolution/Anti-Inflammatory, Neuroinflammation/Neurodegenerative conditions, non-alcoholic steatohepatitis (NASH) and/or non-alcoholic fatty liver disease (NAFLD) (Fatty Liver) & Metabolic Associated Hepatic Dysfunction, Pulmonary Fibrosis & pulmonary dysfunction, and Renal dysfunction that require Nephroprotective measures.

17. A method of treating an inflammatory condition comprising:
a. providing a composition comprising cannabinoid, beta-caryophyllene (BCP), and Vitamin A; and
b. administering an effective amount of the composition to the subject to act on the subject's inflammatory condition.

18. The method of claim 17, further comprising significantly increasing the cellular antioxidant capacity of the subject.

19. The method of claim 17, further comprising significantly increasing the Superoxide dismutase 2 protein (SOD2) activity of the subject.

20. The method of claim 17, further comprising significantly increasing the binding activity of PPAR-gamma in the subject.
